(19) United States
(12) Patent Application Publication
(10) Pub. No.: US 2008/0221321 A1
(43) Pub. Date: Sep. 11, 2008

(54) Preparing Salts of Dihydroxychlorotriazine

(75) Inventors: Stephanie Schauhoff, Langen (DE); Frank Kleff, Brunchkoebel-Oberissigheim (DE)

Correspondence Address: OBLON, SPIVAK, MCCLELLAND MAIER & NEUSTADT, P.C., 1940 DUKE STREET, ALEXANDRIA, VA, 22314, US

(73) Assignee: DEGUSSA GmbH, Duesseldorf (DE)

(21) Appl. No.: 11/911,763

(22) PCT Filed: Apr. 4, 2006
(86) PCT No.: PCT/EP2006/061294

§ 371 Date: Oct. 17, 2007

(30) Foreign Application Priority Data
Apr. 21, 2005 (DE) 10 2005 018 500.2

(51) Int. Cl. C07D 251/28

(52) U.S. Cl. 544/190

(57) ABSTRACT

The present invention is directed to a process for preparing salts of dihydroxychlorotriazine. The process comprises the hydrolysis of cyanuric chloride in alkaline aqueous solution and subsequent crystallization of monosalt of dihydroxychlorotriazine at neutral pH. The crystallization is improved with so-called dispersing assistants.

The present invention is directed to a process for preparing salts of dihydroxychlorotriazine (DHCT). The process of the present invention is effected by partial hydrolysis of cyanuric chloride (CYC) in aqueous solution with subsequent crystallization of the monosalt of the DHCT.

Dihydroxychlorotriazine (DHCT) and its salts are important intermediates in organic synthesis, for example for preparing fire-retardant compounds used in cellulose-based textile materials of construction (DE 10155066).

GB896814 describes various ways to prepare salts of DHCT which proceed from a CYC suspension into which an NaOH solution is metered. The disadvantage with this approach is that acetone is used to prepare the CYC suspension in water, i.e. the reaction is carried out in water/acetone. The reported reaction temperatures are <=30° C. A purity is reported, but it is not mentioned how it was determined and what the by-products are (residual water content/salts or organic by-products?). The quality of the end product is therefore difficult to gauge from the patent's data.

Horrobin et al. report in J. Chem. Soc. 1963, 8, 4130-45 on “The hydrolysis of some chloro-1,3,5-triazines: mechanism, structure and reactivity”.

This paper describes a further way to synthesize the sodium salt of DHCT, namely by adding CYC powder to an NaOH solution. The solution prepared is only 4.6% strong, which does not present any problems with regard to the precipitation and isolation of the DHCT salt at this concentration.

EP 0 597 312 A1 concerns gelatin hardening with triazine derivatives. The DHCT monosodium salt synthesis described is similar to Horrobin et al. A merely 4.6% solution is prepared, and the product is not isolated but further diluted for use.

Against the background of the poor product purities and low concentrations of the solutions used, the processes described appear to be disadvantageous for practice on a large industrial scale.

It is an object of the present invention to provide a further process for preparing salts of dihydroxychlorotriazine that appears to be suitable, in particular, for manufacturing these compounds on an industrial scale. The subject process shall be economically and ecologically superior to prior art processes. The process shall ensure the production of the dihydroxychlorotriazine salts in very high yields and purities and shall ideally not require any further organic solvents to be carried out.

We have found that this object and further objects, unspecified but obvious from the prior art, are achieved by a process having the features of the subject claim 1. Preferred embodiments of the process of the present invention are claimed in claim 1's appendant subclaims 2 to 4.

A process for preparing a salt of dihydroxychlorotriazine by partial hydrolysis of cyanuric chloride in aqueous solution and subsequent precipitation in a pH range at which the monosalt of dihydroxychlorotriazine is predominant, wherein the precipitation is carried out in the presence of an organic dispersing assistant, is a very simple yet advantageous way to achieve the stated object. Adding an organic dispersing assistant in the crystallization of a monosalt of dihydroxychlorotriazine obviates, very surprisingly, the use of otherwise necessary organic solvents without the yield of these salts or their purity being adversely affected as a result. On the contrary, the process of the present invention further enhances the purity and yield compared with prior art data, which is attributable to the fact that a readily crystallizing solid is obtained through the dispersing assistant added. Without the addition of dispersing assistant, the Ciba approach (neutralization at R.T., but without acetone or prior reprecipitation) leads, in particular at the high concentrations chosen, to the as-precipitated dihydroxychlorotriazine being so gooey that the mixture is no longer stirrable. As a result, some spots become over-acidified in the course of further neutralization, giving rise to larger amounts of by-products.

From the monosalts, other mono- or disalts are readily preparable in further steps at the discretion of a person skilled in the art.

A person skilled in the art knows which dispersing assistants come into consideration for the process of the present invention. Typical dispersing assistants are obtainable for example from Cognis, Clariant, Goldschmidt or BASF. Preference is given to using dispersing assistants selected from the group consisting of
polyacrylic salts
fatty acid esters.

Very particular preference is given to the use of fatty acid ester ethoxylates, fatty acid polyglycol esters and polyacrylic acid salts. It is extremely preferable to use fatty acid polyglycol ester having 4-15 EO units as dispersing assistant in the subject reaction.

A preferred way to carry out the process of the present invention is for cyanuric chloride to be metered into an aqueous solution and alkalized with an inorganic base. It is now advantageous for the aqueous solution to be admixed with a buffering system before the cyanuric chloride is added in order that strong pH swings at the start of the reaction and hence the formation of by-products may be avoided. Advantageous buffering systems include those which buffer in the pH range of 8-9. It is very particularly advantageous to buffer the aqueous solution with NaHCO₃.

The amount of buffering system added is at the discretion of a person skilled in the art. Based on the cyanuric chloride, an amount of 5-30 mol % is advantageous, of 10-25 mol % is preferable and of 15-20 mol % is most preferable.

During the addition of the cyanuric chloride or subsequently to its addition, the pH of the reaction mixture is maintained/set at ranges from 11.5 to 13.5, preferably 12 to 13 and most preferably of 12.5±0.2. The simultaneous addition of base and cyanuric chloride permits an efficiently controllable reaction and a safe procedure on a manufacturing scale and similarly prevents the formation of by-products, which is why this approach is preferable.

The pH can be set with inorganic bases comprising the later cation in the monosalt of dihydroxychlorotriazine.

Alkali metals are preferred as cations and Na⁺ is very particularly preferred in this connection.

Hydroxide is used as base. Sodium hydroxide is particularly preferred.

The hydrolysis preferably takes place in a temperature interval where hydrolysis is sufficiently rapid and where the risk of full hydrolysis to by-produce 2,4,6-trihydroxy-1,2,3-triazine (cyanuric acid) is optimally calibrated out. In the given circumstances, the hydrolysis temperature is advantageously set between 20° C. and 60° C., preferably between 30° C. and 50° C. and most preferably to 40±5° C.

To precipitate the monosalt of dihydroxychlorotriazine, the dispersing assistant is then added to the solution and subsequently the pH of the solution is set to a range at which the monosalt of DHCT is predominant. Predominant in this context means >50 mol %, preferably >70 mol %, more preferably >80 mol % and most preferably >90 mol % compared with the sum total of the other derivatives of DHCT present at equilibrium. This is advantageously the case in a pH range of 6.5 to 8.5, preferably 7 to 8 and most preferably 7.5±0.2. Acidifying can be effected using preferably inorganic acids known to a skilled person for this purpose, examples being hydrochloric acid, sulphuric acid and phosphoric acid. It is very particularly preferable to use hydrochloric acid for this purpose.

The solution or the resulting suspension is subsequently cooled down to values in the range from 0 to 25° C., preferably in the range from 5 to 20° C. and most preferably in the range of 12.5±2.5° C. After crystallization has ended, the product can be filtered off and dried. The dried solid contains 75 to 80 percent of pure monosalt of dihydroxychlorotriazine.

As mentioned earlier, the dihydroxychlorotriazine monosalt thus obtained can be converted into another monosalt or into the corresponding disalt.

The subject process makes it possible to obtain efficiently filterable monosalts of dihydroxychlorotriazine in excellent purity and with high yield compared with prior art processes. Reworking the process presented in the Ciba patent discussed at the beginning gives a dried solid that includes a mere 73 percent of pure monosalt of dihydroxychlorotriazine. The yield of salt based on cyanuric chloride is only 79 percent.

The process of the present invention, by contrast, leads to solids that include 75 to 80 percent of monosalt of dihydroxychlorotriazine. Further constituents of the solid are very substantially by-product-free neutralization salts and water. The yield in this instance is in the range from 80% to 90% based on cyanuric chloride used. Organic impurities such as cyanuric acid for example are, as mentioned only present in a minor amount.

It is a further achievement of the process of the present invention that the cyanuric chloride can be reacted in pure water without prior treatment (dissolving/precipitating) and without addition of an organic solvent, which is more cost effective, environment friendlier and technically simpler. Moreover, achieving the abovementioned higher yield requires no supplementary precipitation and no addition of salt; or even higher yields could be achieved, if appropriate, by adding salt.

Aqueous solution in the sense of the present invention is to be understood as meaning a solution which includes water as main constituent, but which may also additionally comprise further water-soluble organic solvents. Organic solvents inert to the reactants are advantageous, examples being ketones (acetone, MIBK) and ethers (THF, DME). It is extremely preferable to use water without addition of further organic solvents.

### EXAMPLES

### Example 1

615.7 g of water are admixed with 8.3 g of NaHCO₃ and heated to 40° C. 18.4 g (0.1 mol) of CYC are added in solid form and suspended. Then, 20% aqueous sodium hydroxide solution is added under pH control such that the pH is maintained between 12.3 and 12.7. At intervals of 5 min, the rest of the CYC is added in 18.4 g portions during the metered addition of the NaOH. The entire CYC addition (5×18.4 g) accordingly takes place over a period of about 25 min. Further NaOH solution is subsequently added until the pH remains constant at about 12.5. In total, 337.9 g of 20% NaOH solution are needed for the reaction.

| Reaction time: | 1.5 h |
|---|---|
| CYC conversion: | 100% |

Precipitation of product: The solution is admixed with 0.01% by weight of Genagen O 060. The pH of the temperature-controlled solution at 40° C. is gradually adjusted to 7.5 with concentrated HCl. The solution is subsequently cooled down to 10° C., subsequently stirred for 1 hour before the product is filtered off with suction and dried at 60° C./10 mbar.

| Filtration time: | <3 min |
|---|---|
| Isolation yield based on CYC: | 104.7% |
| Composition: | 84.8% of DHCT-Na |
| | 0.1% of cyanuric acid |
| | 6.5% of water |
| | 8.6% of salt content |
| Na DHCT yield based on CYC: | 88.7% |

### Comparative Example 1

### Low Concentration

Preparation of disodium salt as in Example 1 of Ciba patent.

| Reaction time: | 4 h |
|---|---|
| CYC conversion: | 100% |

Precipitation of product as in Ciba patent: The solution is adjusted at R.T. to pH 7 with 10 N HCl, and cooled down to 10° C., with stirring. After filtration, further product is supplementarily precipitated by addition of 10 g of NaCl to 100 g of solution. Drying takes place at 60° C./10 mbar.

| Filtration time: | <3 min |
|---|---|
| Isolation yield based on CYC: | 108.6% |
| Composition: | 72.9% of DHCT-Na |
| | 0.1% of cyanuric acid |
| | 14.7% of water |
| | 12.3% of salt content |
| Na DHCT yield based on CYC: | 79.2% |

### Comparative Example 2

### Comparable Concentration

Preparation of disodium salt in a concentration as in our Example 1, but precipitation as in Example 1 of Ciba patent.

| Reaction time: | 1.5 h |
|---|---|
| CYC conversion: | 100% |

Precipitation of product similarly to Ciba patent: The solution is adjusted at R.T. to pH 7 with 10 N HCl, and cooled down to 10° C., with stirring. After filtration, further product is supplementarily precipitated by addition of 10 g of NaCl to 100 g of solution. Drying takes place at 60° C./10 mbar.

| Filtration time: | 11 min (gooey consistency) |
|---|---|
| Isolation yield based on CYC: | 114.4% |
| Composition: | 76.6% of DHCT-Na |
| | 0.5% of cyanuric acid |
| | 12.1% of water |
| | 10.8% of salt content |
| Na DHCT yield based on CYC: | 87.6% |

### Comparative Example 3

Preparation of disodium salt in a concentration as in our Example 1, precipitation also as in our Example 1, but without dispersing assistant.

| Reaction time: | 1.5 h |
|---|---|
| CYC conversion: | 100% |

Precipitation of product: The pH of the temperature-controlled solution at 40° C. is gradually adjusted to 7.5 with concentrated HCl. The solution is subsequently cooled down to 10° C., subsequently stirred for 1 hour before the product is filtered off with suction and dried at 60° C./10 mbar.

| Filtration time: | 10 min (gooey consistency) |
|---|---|
| Isolation yield based on CYC: | 108.5% |
| Composition: | 81.4% of DHCT-Na |
| | 0.4% of cyanuric acid |
| | 8.2% of water |
| | 10% of salt content |
| Na DHCT yield based on CYC: | 88.3% |

1-4. (canceled)

5. A process for preparing a dihydroxychlorotriazine (DHCT) salt comprising:
partially hydrolyzing cyanuric chloride (CYC) in an aqueous solution; and
precipitating the DHCT salt in the presence of at least one organic dispersing assistant.

6. The process for preparing a DHCT salt according to claim 5, wherein said precipitating is carried out in a pH range at which precipitation of a DHCT monosalt is predominant.

7. The process for preparing a DHCT salt according to claim 5, wherein said organic dispersing assistant is at least one selected from the group consisting of polyacrylic salts, fatty acid esters, and combinations thereof.

8. The process for preparing a DHCT salt according to claim 5, wherein said organic dispersing assistant is one or more polyacrylic salts.

9. The process for preparing a DHCT salt according to claim 5, wherein said organic dispersing assistant is one or more fatty acid esters.

10. The process for preparing a DHCT salt according to claim 5 further comprising buffering said aqueous solution with a buffer prior to said partially hydrolyzing.

11. The process for preparing a DHCT salt according to claim 10, wherein said buffer is sodium bicarbonate.

12. The process for preparing a DHCT salt according to claim 10, wherein said buffer is present in an amount ranging from 5 mol % to 30 mol % relative to CYC.

13. The process for preparing a DHCT salt according to claim 12, wherein said buffer is present in an amount ranging from 10 mol % to 25 mol % relative to CYC.

14. The process for preparing a DHCT salt according to claim 13, wherein said buffer is present in an amount ranging from 15 mol % to 20 mol % relative to CYC.

15. The process for preparing a DHCT salt according to claim 5, wherein said partially hydrolyzing is carried out at a temperature ranging from 20° C. to 60° C.

16. The process for preparing a DHCT salt according to claim 15, wherein said partially hydrolyzing is carried out at a temperature ranging from 30° C. to 50° C.

17. The process for preparing a DHCT salt according to claim 16, wherein said partially hydrolyzing is carried out at a temperature ranging from 35° C. to 45° C.

18. The process for preparing a DHCT salt according to claim 6, wherein said pH range is from 6.5 to 8.5.

19. The process for preparing a DHCT salt according to claim 18, wherein said pH range is from 7 to 8.

20. The process for preparing a DHCT salt according to claim 19, wherein said pH range is from 7.3 to 7.7.

21. The process for preparing a DHCT salt according to claim 6, wherein said DHCT monosalt is predominantly present in an amount of greater than 50 mol % relative to other DHCT derivatives.

22. The process for preparing a DHCT salt according to claim 21, wherein said DHCT monosalt is predominantly present in an amount of greater than 70 mol % relative to other DHCT derivatives.

23. The process for preparing a DHCT salt according to claim 22, wherein said DHCT monosalt is predominantly present in an amount of greater than 80 mol % relative to other DHCT derivatives.

24. The process for preparing a DHCT salt according to claim 23, wherein said DHCT monosalt is predominantly present at a concentration of greater than 90 mol % relative to other DHCT derivatives.
